(19) United States
(12) Patent Application Publication
(10) Pub. No.: US 2009/0248124 A1
(43) Pub. Date: Oct. 1, 2009

(54) LEAD IDENTIFIER FOR AN IMPLANTABLE ELECTRIC STIMULATION SYSTEM AND METHODS OF MAKING AND USING

(75) Inventors: Anne Margaret Pianca, Santa Monica, CA (US); Aditya Vasudeo Pandit, Burbank, CA (US); Sarvani Grandhe, Santa Clarita, CA (US); Aaron James Gifford, Lake Elsinore, CA (US); Kenny Kinyen Chinn, Castaic, CA (US)

Correspondence Address: Boston Scientific Neuromodulation Corp.;c/o DARBY & DARBY P.C., P.O. BOX 770, Church Street Station, NEW YORK, NY, 10008-0770, US

(73) Assignee: Boston Scientific Neuromodulation Corporation, Valencia, CA (US)

(21) Appl. No.: 12/412,104

(22) Filed: Mar. 26, 2009

Related U.S. Application Data
(60) Provisional application No. 61/040,098, filed on Mar. 27, 2008.

(51) Int. Cl. A61N 1/05

(52) U.S. Cl. 607/116

(57) ABSTRACT

A lead includes a lead body with a distal end and at least one proximal end. The lead includes at least one lead identifier disposed on the lead body. The lead identifier is configured and arranged to visually identify the end of the lead body on which the at least one lead identifier is disposed. The at least one lead identifier includes at least one of a markable-surface-finish region suitable for marking with a pen, at least one laser-ablated identification marking, at least one contrasting band of material formed of a conspicuous color, at least one conspicuously-colored spacer disposed between two adjacent terminals of at least one of the at least one proximal end of the lead body, or a conspicuously-colored proximal tip disposed on at least one of the at least one proximal end of the lead body.

### CROSS-REFERENCE TO RELATED APPLICATIONS

This application is a utility patent application based on a previously filed U.S. Provisional Patent Application Ser. No. 61/040,098 filed on Mar. 27, 2008, the benefit of which is hereby claimed under 35 U.S.C. §119(e) and incorporated herein by reference.

### TECHNICAL FIELD

The present invention is directed to the area of implantable electrical stimulation systems and methods of making and using the systems. The present invention is also directed to implantable electrical stimulation systems that include one or more leads with lead identifiers for identifying the leads, as well as methods of making and using the lead identifiers, leads, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Deep brain stimulation has also been useful for treating refractory chronic pain syndromes and has been applied to treat movement disorders and epilepsy. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Moreover, electrical stimulation systems can be implanted subcutaneously to stimulate subcutaneous tissue including subcutaneous nerves such as the occipital nerve.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a lead includes a lead body with a distal end and at least one proximal end. The lead also includes a plurality of electrodes disposed on the distal end of the lead body and a plurality of terminals disposed on the at least one proximal end of the lead body. A plurality of conductor wires extend along the lead body and couple the electrodes electrically to the terminals. The lead further includes at least one lead identifier disposed on the lead body. The lead identifier is configured and arranged to visually identify the end of the lead body on which the at least one lead identifier is disposed. The at least one lead identifier includes at least one of a markable-surface-finish region suitable for marking with a pen, at least one laser-ablated identification marking, at least one contrasting band of material formed of a conspicuous color, at least one conspicuously-colored spacer disposed between two adjacent terminals of at least one of the at least one proximal end of the lead body, or a conspicuously-colored proximal tip disposed on at least one of the at least one proximal end of the lead body.

In another embodiment, an electrical stimulating system includes a first lead, a control module configured and arranged to electrically couple to the first lead, and a connector for receiving the first lead. The first lead includes a lead body with a distal end and at least one proximal end. The lead also includes a plurality of electrodes disposed on the distal end of the lead body and a plurality of terminals disposed on the at least one proximal end of the lead body. A plurality of conductor wires extend along the lead body and couple the electrodes electrically to the terminals. The lead further includes at least one lead identifier disposed on the lead body. The lead identifier is configured and arranged to visually identify the end of the lead body on which the at least one lead identifier is disposed. The at least one lead identifier includes at least one of a markable-surface-finish region suitable for marking with a pen, at least one laser-ablated identification marking, at least one contrasting band of material formed of a conspicuous color, at least one conspicuously-colored spacer disposed between two adjacent terminals of at least one of the at least one proximal end of the lead body, or a conspicuously-colored proximal tip disposed on at least one of the at least one proximal end of the lead body. The control module includes a housing and an electronic subassembly disposed in the housing. The connector includes a connector housing defining at least one opening for receiving the at least one proximal end of the first lead, and a plurality of connector contacts disposed in the connector housing. The connector contacts are configured and arranged to couple to at least one terminal on the at least one proximal end of the first lead.

In yet another embodiment, a method for stimulating patient tissue includes implanting at least one lead with a plurality of electrodes into a patient, disposing each of at least one proximal end of the at least one lead into at least one connector electrically coupled to at least one control module, and providing electrical signals from at least one of the control modules to electrically stimulate patient tissue using at least one of the plurality of electrodes. The at least one lead including a plurality of terminals disposed on each of at least one proximal end of each of the at least one lead, each of the plurality of terminals electrically coupled to at least one of the plurality of electrodes. Each of the at least one lead also including at least one lead identifier for individually identifying each of the at least one proximal ends of the at least one lead. The at least one lead identifier includes at least one of a markable-surface-finish region suitable for marking with a pen, at least one laser-ablated identification marking, at least one contrasting band of material formed of a conspicuous color, at least one conspicuously-colored spacer disposed between two adjacent terminals of at least one of the at least one proximal end of the lead body, or a conspicuously-colored proximal tip disposed on at least one of the at least one proximal end of the lead body. Each of the at least one connector defining at least one port for receiving the at least one proximal ends of the at least one lead. Each of the at least one port including a plurality of connective contacts that electrically couple to at least one of the plurality of terminals disposed on each of at least one proximal ends of each of the at least one lead. Each of the at least one the connectors electrically coupled to at least one control module.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2 is a schematic view of another embodiment of an electrical stimulation system, according to the invention;

FIG. 3A is a schematic view of one embodiment of a proximal portion of a lead and a control module of an electrical stimulation system, according to the invention;

FIG. 3B is a schematic view of one embodiment of a proximal portion of a lead and a lead extension for an electrical stimulation system, according to the invention;

FIG. 4A is a schematic side view of one embodiment of a proximal end of a lead with a lead identifier that includes a markable surface finish disposed on an outer surface of the lead, according to the invention;

FIG. 4B is a schematic side view of one embodiment of the proximal end of the lead shown in FIG. 4A with a first pen marking on the markable surface finish, according to the invention;

FIG. 4C is a schematic side view of one embodiment of the proximal end of the lead shown in FIG. 4A with a second pen marking on the markable surface finish, according to the invention;

FIG. 4D is a schematic side view of one embodiment of the proximal end of the lead shown in FIG. 4A with a third pen marking on the markable surface finish, according to the invention;

FIG. 5A is a schematic side view of one embodiment of a proximal end of a lead with a lead identifier that includes a laser-ablated identification code disposed on an outer surface of the lead, according to the invention;

FIG. 5B is a schematic side view of one embodiment of a proximal end of a lead with a lead identifier that includes a laser-ablated band disposed on an outer surface of the lead, according to the invention;

FIG. 5C is a schematic side view of one embodiment of a proximal end of a lead with a lead identifier that includes a laser-ablated identification code and laser-ablated bands disposed on an outer surface of the lead, according to the invention;

FIG. 6A is a schematic perspective view of one embodiment of a proximal end of a lead with a lead identifier that includes a contrasting band of material wrapped over a portion of a lateral circumference of an outer surface of the lead, according to the invention;

FIG. 6B is a schematic side view of one embodiment of a proximal end of a lead with a lead identifier that includes two contrasting bands of material wrapped over at least a portion of a lateral circumference of an outer surface of the lead, according to the invention;

FIG. 6C is a schematic side view of one embodiment of a proximal end of a lead with a lead identifier that includes four contrasting bands of material of different widths wrapped over at least a portion of a lateral circumference of an outer surface of the lead, according to the invention;

FIG. 6D is a schematic side view of one embodiment of a proximal end of a lead with a lead identifier that includes two contrasting bands of material with markings wrapped over at least a portion of a lateral circumference of an outer surface of the lead, according to the invention;

FIG. 6E is a schematic side view of one embodiment of a proximal end of a lead with a lead identifier that includes two contrasting bands of material wrapped over at least a portion of a lateral circumference of an inset region disposed in an outer surface of the lead, according to the invention;

FIG. 6F is a schematic side view of one embodiment of a proximal end of a lead with a lead identifier that includes two contrasting bands of material with markings wrapped over at least a portion of a lateral circumference of an inset region disposed in an outer surface of the lead, according to the invention;

FIG. 7A is a schematic side view of one embodiment of a proximal end of a lead with a lead identifier that includes a conspicuously-colored spacer disposed between two adjacent terminals, according to the invention;

FIG. 7B is a schematic side view of one embodiment of a proximal end of a lead with a lead identifier that includes seven conspicuously-colored spacers disposed between adjacent terminals, according to the invention;

FIG. 7C is a schematic side view of one embodiment of a proximal end of a lead with a lead identifier that includes a conspicuously-colored proximal tip, according to the invention;

FIG. 7D is a schematic side view of one embodiment of a proximal end of a lead with a lead identifier that includes three conspicuously-colored spacers disposed between adjacent terminals and a conspicuously-colored proximal tip, according to the invention;

FIG. 8A is a schematic side view of one embodiment of a proximal end of a lead with a lead identifier that includes a conspicuously-colored proximal tip, a markable surface finish marked with two dots, and a laser-ablated band, according to the invention;

FIG. 8B is a schematic side view of one embodiment of a proximal end of a lead with a lead identifier that includes a laser-ablated identification code and a contrasting band of material wrapped around at least a portion of a lateral circumference of the lead, according to the invention;

FIG. 8C is a schematic side view of one embodiment of a proximal end of a lead with a lead identifier that includes a conspicuously-colored proximal tip and two contrasting bands of material with markings wrapped over at least a portion of a lateral circumference of an inset region disposed in an outer surface of the lead, according to the invention;

FIG. 8D is a schematic side view of one embodiment of a proximal end of a lead with a lead identifier that includes three conspicuously-colored spacers disposed between adjacent terminals, a laser-ablated band, and a contrasting band of material wrapped around a portion of a lateral circumference of the lead, according to the invention;

FIG. 9 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to implantable electrical stimulation systems that include one or more leads with lead identifiers for identifying the leads, as well as methods of making and using the lead identifiers, leads, and electrical stimulation systems.

Suitable implantable electrical stimulation systems include, but are not limited to, an electrode lead (“lead”) with one or more electrodes disposed on a distal end of the lead and one or more terminals disposed on one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; and 6,741,892; and U.S. patent applications Ser. Nos. 10/353,101, 10/503,281, 11/238,240; 11/319,291; 11/327,880; 11/375,638; 11/393,991; and 11/396,309, all of which are incorporated by reference.

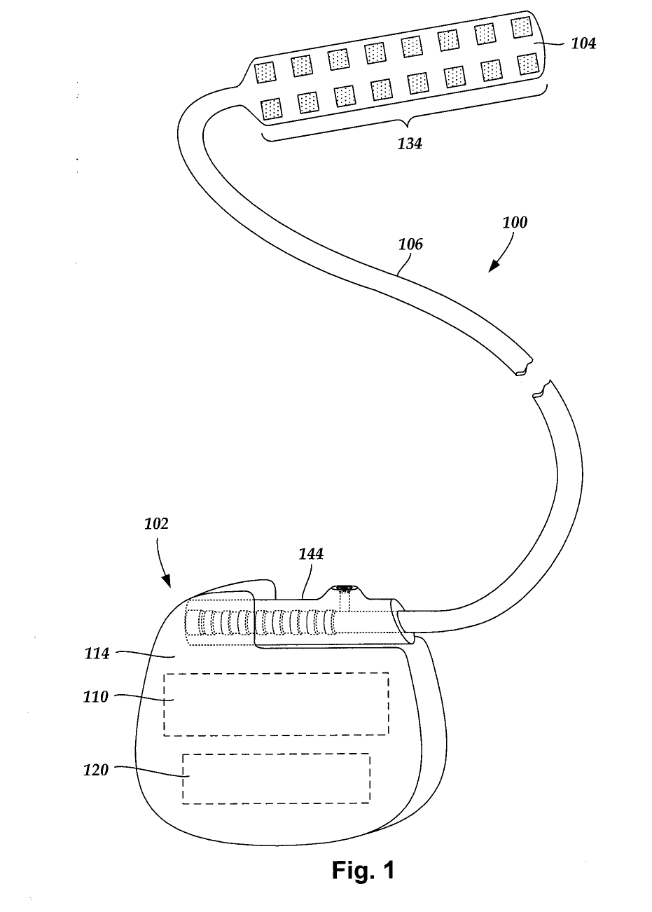

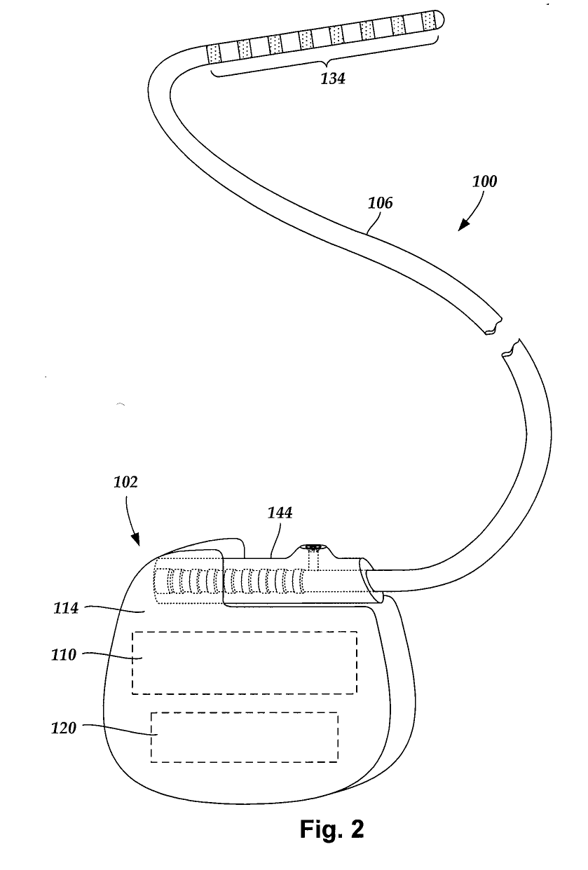

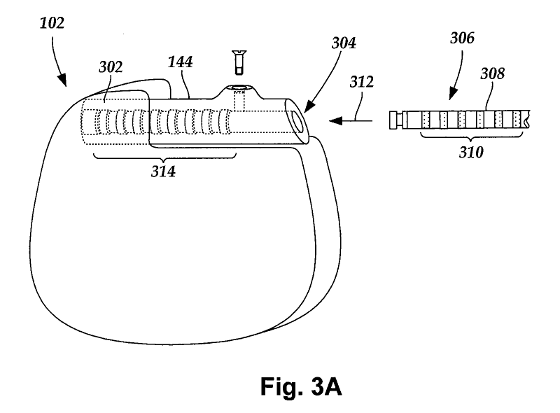

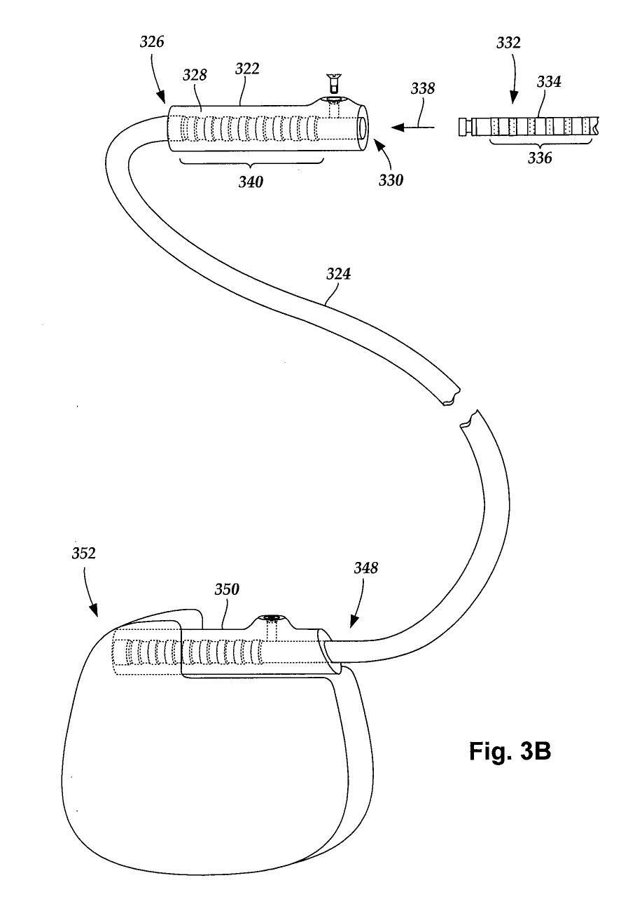

FIG. 1 illustrates schematically one embodiment of an electrical stimulation system 100. The electrical stimulation system includes a control module (e.g., a stimulator or pulse generator) 102, a paddle body 104, and at least one lead body 106 coupling the control module 102 to the paddle body 104. The paddle body 104 and the one or more lead bodies 106 form a lead. The paddle body 104 typically includes an array of electrodes 134. The control module 102 typically includes an electronic subassembly 110 and an optional power source 120 disposed in a sealed housing 114. The control module 102 typically includes a connector 144 (FIGS. 2 and 3A, see also 322 and 350 of FIG. 3B) into which the proximal end of the one or more lead bodies 106 can be plugged to make an electrical connection via conductive contacts on the control module 102 and terminals (e.g., 310 in FIG. 3A and 336 of FIG. 3B) on each of the one or more lead bodies 106. It will be understood that the electrical stimulation system can include more, fewer, or different components and can have a variety of different configurations including those configurations disclosed in the electrical stimulation system references cited herein. For example, instead of a paddle body 104, the electrodes 134 can be disposed in an array at or near the distal end of the lead body 106 forming a percutaneous lead, as illustrated in FIG. 2. A percutaneous lead may be isodiametric along the length of the lead. In addition, one or more lead extensions 312 (see FIG. 3B) can be disposed between the one or more lead bodies 106 and the control module 102 to extend the distance between the one or more lead bodies 106 and the control module 102 of the embodiments shown in FIGS. 1 and 2.

The electrical stimulation system or components of the electrical stimulation system, including one or more of the lead bodies 106, the paddle body 104, and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. The number of electrodes 134 in the array of electrodes 134 may vary. For example, there can be two, four, six, eight, ten, twelve, fourteen, sixteen, or more electrodes 134. As will be recognized, other numbers of electrodes 134 may also be used.

The electrodes of the paddle body 104 or one or more lead bodies 106 are typically disposed in, or separated by, a non-conductive, biocompatible material including, for example, silicone, polyurethane, polyetheretherketone (“PEEK”), epoxy, and the like or combinations thereof. The paddle body 104 and one or more lead bodies 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 310 in FIG. 3A and 336 of FIG. 3B) are typically disposed at the proximal end of the one or more lead bodies 106 for connection to corresponding conductive contacts (e.g., 314 in FIG. 3A and 340 of FIG. 3B) in connectors (e.g., 144 in FIGS. 1-3A and 322 and 350 of FIG. 3B) disposed on, for example, the control module 102 (or to other devices, such as conductive contacts on a lead extension, an operating room cable, or an adaptor). Conductive wires (not shown) extend from the terminals (e.g., 310 in FIG. 3A and 336 of FIG. 3B) to the electrodes 134. Typically, one or more electrodes 134 are electrically coupled to a terminal (e.g., 310 in FIG. 3A and 336 of FIG. 3B). In some embodiments, each terminal (e.g., 310 in FIG. 3A and 336 of FIG. 3B) is only connected to one electrode 134. The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet rod to facilitate placement of the lead within a body of a patient. Additionally, there may also be one or more lumens (not shown) that open at, or near, the distal end of the lead, for example, for infusion of drugs or medication into the site of implantation of the paddle body 104. In at least one embodiment, the one or more lumens may be flushed continually, or on a regular basis, with saline, epidural fluid, or the like. In at least some embodiments, the one or more lumens can be permanently or removably sealable at the distal end.

In at least some embodiments, leads are coupled to connectors disposed on control modules. In FIG. 3A, a lead 308 is shown configured and arranged for insertion to the control module 102. The connector 144 includes a connector housing 302. The connector housing 302 defines at least one port 304 into which a proximal end 306 of a lead 308 with terminals 310 can be inserted, as shown by directional arrow 312. The connector housing 302 also includes a plurality of conductive contacts 314 for each port 304. When the lead 308 is inserted into the port 304, the conductive contacts 314 can be aligned with the terminals 310 on the lead 308 to electrically couple the control module 102 to the electrodes (134 of FIG. 1) disposed at a distal end of the lead 308. Examples of connectors in control modules are found in, for example, U.S. Pat. No. 7,244,150 and U.S. patent application Ser. No. 11/532,844, which are incorporated by reference.

In FIG. 3B, a connector 322 is disposed on a lead extension 324. The connector 322 is shown disposed at a distal end 326 of the lead extension 324. The connector 322 includes a connector housing 228. The connector housing 228 defines at least one port 330 into which a proximal end 332 of a lead 334 with terminals 336 can be inserted, as shown by directional arrow 338. The connector housing 328 also includes a plurality of conductive contacts 340. When the lead 334 is inserted into the port 330, the conductive contacts 340 disposed in the connector housing 328 can be aligned with the terminals 336 on the lead 334 to electrically couple the lead extension 324 to the electrodes (134 of FIG. 1) disposed at a distal end (not shown) of the lead 334.

In at least some embodiments, the proximal end of a lead extension is similarly configured and arranged as a proximal end of a lead. The lead extension 324 may include a plurality of conductive wires (not shown) that electrically couple the conductive contacts 340 to a proximal end 348 of the lead extension 324 that is opposite to the distal end 326. In at least some embodiments, the conductive wires disposed in the lead extension 324 can be electrically coupled to a plurality of terminals (not shown) disposed on the proximal end 348 of the lead extension 324. In at least some embodiments, the proximal end 348 of the lead extension 324 is configured and arranged for insertion into a connector disposed in another lead extension. In other embodiments, the proximal end 348 of the lead extension 324 is configured and arranged for insertion into a connector disposed in a control module. As an example, in FIG. 3B the proximal end 348 of the lead extension 324 is inserted into a connector 350 disposed in a control module 352.

During implantation of a plurality of leads into a patient, or implantation of a lead with multiple proximal ends into a patient, once the distal end(s) are positioned, each proximal end of the one or more leads is typically coupled to a specific port defined in a specific connector of a control module, a lead extension, an adaptor, or an operating room cable. When a plurality of leads or a plurality of proximal ends of one lead is present, determining which proximal end should be coupled to which port can sometimes be difficult or time-consuming. It may be the case that the proximal ends of the one or more leads may be visually and fluoroscopically identical to one another and the distal end of each of the one or more leads may be visually obscured. Moreover, multiple proximal ends may be inserted through a single subcutaneous tunnel and may become overlapped or tangled with each other or various body tissues. Similar proximal-end-identification problems may also exist during revision surgery, for example, during explantation of a malfunctioning lead. It may be a time-consuming task to identify the malfunctioning lead.

In at least some embodiments, a lead identifier is disposed on one or more leads, preferably at or near the proximal ends of the lead, to visually identify (and preferably uniquely identify) the lead. Lead identifiers can be used, for example, to distinguish the proximal end on which the lead identifier is disposed from other proximal ends of the same lead or from proximal ends on different leads to facilitate the coupling of the identified proximal end of the lead to a desired corresponding port of a control module or a lead extension.

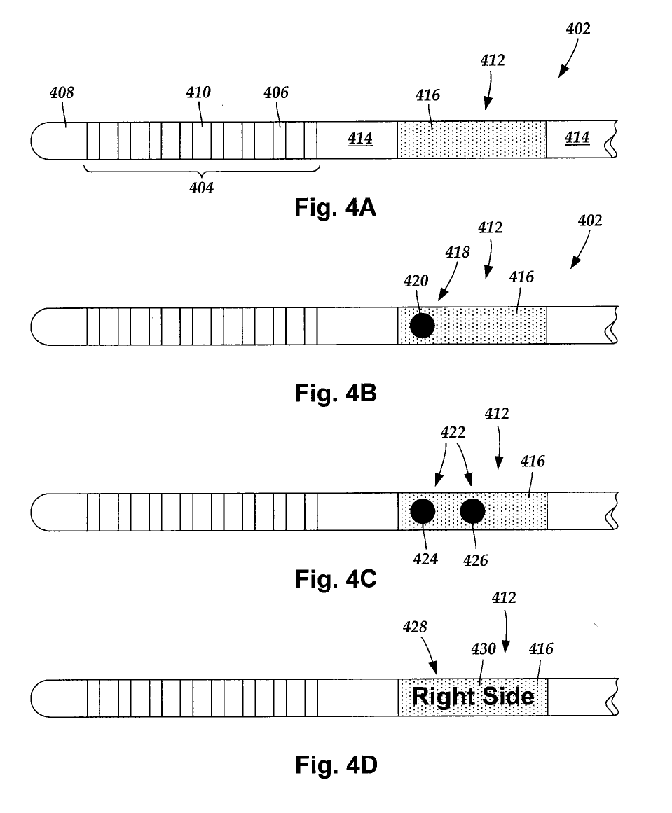

In some embodiments, a lead identifier includes at least one markable region with a surface finish different from other portions of the lead. FIG. 4A is a schematic side view of one embodiment of a proximal end of a lead 402 with a plurality of terminals 404, such as terminal 406, and a proximal tip 408. Each adjacent terminal is separated by a spacer, such as spacer 410. The lead 402 further includes a lead identifier 412 disposed on an outer surface 414 of the lead 402. In FIG. 4A, the lead identifier 412 includes a markable surface finish 416. The surface finish 416 may be formed in the desired shape by any suitable process including, for example, laser ablation, grit blasting, etching, grinding, machining, and the like. The surface finish may, for example, roughen the surface to provide surface features that can capture and hold ink, such as by adsorption.

In a preferred embodiment, a markable surface finish is formed by laser ablation and is suitable for marking with a pen. In at least some embodiments, the markable surface finish can be marked with a pen in order to identify the proximal end of the lead on which the markable surface finish is disposed. In at least some embodiments, the region with the markable surface is at or near a proximal end of the lead. The region may be proximal to, or distal to, or between, the terminals of the lead.

It will be understood that many different possible markings can be formed on the markable surface finish 416 to identify the proximal end of the lead 402. For example, a markable-surface-finish marking may be one or more geometric shapes, lines, numbers, letters, symbols, or pictures. Additionally, markable-surface-finish markings can be identifiable by other determinants, such as size, shape, number of markings, or placement of markings on the markable surface finish 416. For example, FIG. 4B shows a schematic side view of one embodiment of the proximal end of the lead 402 with the lead identifier 412 that includes a first marking 418 on the markable surface finish 416. The first marking 418 includes a dot 420. In another example, FIG. 4C shows the lead identifier 412 with a second marking 422 on the markable surface finish 416. The second marking 422 includes two dots 424 and 426. In yet another example, FIG. 4D shows the lead identifier 412 with a third marking 428 on the markable surface finish 416. The third marking 428 includes the words “Right Side” 430.

In at least some embodiments, a coding system can be implemented in which various markings formed on the markable surface finish correspond with information about the lead on which the markable surface finish is disposed, such as the lead type, the number of proximal ends associated with the lead, which port a proximal end of a lead is to be inserted, the positioning of the distal end of the lead within the anatomy of a patient, or other information. For example, the coding system might indicate a right lead or a left lead or the coding system might indicate a top lead or bottom lead or rostral lead or caudal lead. For example, in one embodiment, a single dot may correspond with a first port defined in a connector of a control module. Similarly, in another embodiment, two dots may indicate that the proximal end of the lead on which the two dots are disposed corresponds with a second port defined in the connector of the control module. In at least some embodiments, a coding system may also be used to differentiate between multiple proximal ends of a single lead.

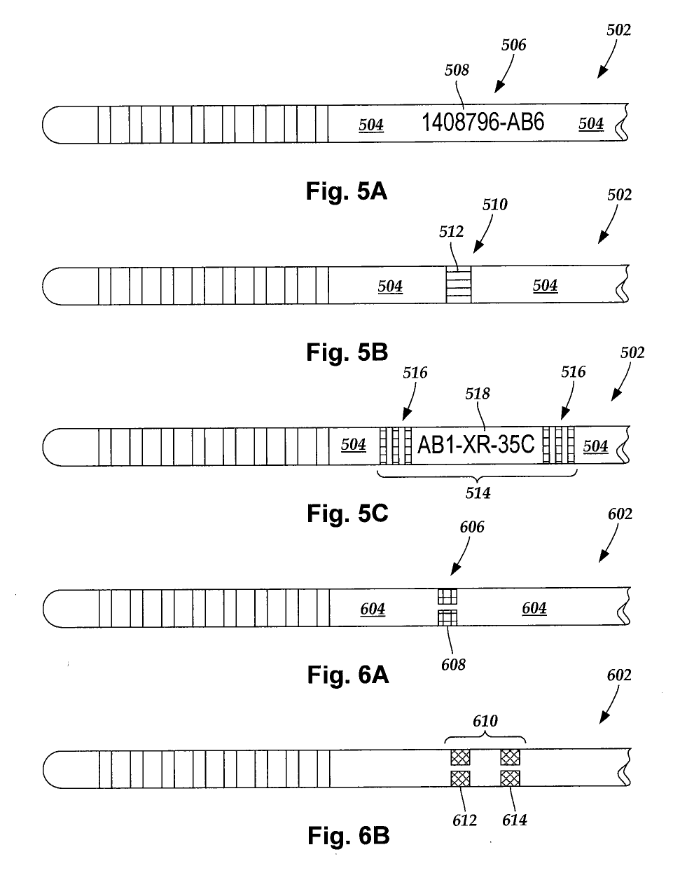

In at least some embodiments, a lead identifier includes at least one identification marking laser-ablated onto an outer surface of a proximal end of a lead. In at least some embodiments, the laser-ablated identification marking includes an identification code. FIG. 5A is a schematic side view of one embodiment of a proximal end of a lead 502 that includes an outer surface 504. The lead 502 further includes a lead identifier 506 disposed on the outer surface 504 that includes a laser-ablated identification code 508. It will be understood that many different laser-ablated identification codes can be used to identify the proximal end of the lead 502. For example, a laser-ablated identification code may be one or more geometric shapes, lines, numbers, letters, symbols, or pictures. Additionally, laser-ablated identification codes can be identifiable by other determinants, such as size, shape, numbers of bits of information, or placement of the laser-ablated identification code on the lead 502. In at least some embodiments, the laser-ablated identification code is the serial number of the lead.

In at least some embodiments, the laser-ablated identification marking includes one or more conspicuous regions. A region can be conspicuous when the region contrasts visually with the portion of the lead on which the conspicuous region is disposed, such that the conspicuous region is visually distinct from the surrounding portions of the lead. Conspicuous laser-ablated regions can be formed in many different regular or irregular shapes, such as an oval, a circle, a rectangle, and the like. In a preferred embodiment, conspicuous laser-ablated regions are formed as one or more bands laser-ablated around a lateral circumference of a proximal end of a lead. In FIG. 5B, the lead 502 includes a lead identifier 510. The lead identifier 510 includes a band 512 laser-ablated onto the outer surface 504 of the lead 502.

Laser-ablated bands can be formed in many different sizes. For example, the laser ablated band 512 can be formed with different widths. The number of laser-ablated bands in a lead identifier also may vary. For example, there can be one, two, three, four, five, six, or more laser-ablated bands. As will be recognized, other numbers of laser-ablated bands may also be used. In alternate embodiments, a lead identifier includes at least one laser-ablated identification code and at least one laser-ablated band. For example, FIG. 5C shows a lead identifier 514 that includes a plurality of laser-ablated bands 516 flanking a laser-ablated identification code 518. In at least some embodiments, the laser-ablated bands are used to increase the conspicuousness of a nearby laser-ablated identification code. In other embodiments, the numbers, sizes, and shapes of the laser-ablated bands can be used as a coding system to provide various types of information about the lead on which the laser-ablated bands are disposed, such as the lead type, the number of proximal ends associated with the lead, which port a proximal end of a lead is to be inserted, the positioning of the distal end of the lead, or other information.

In at least some embodiments, a lead identifier includes at least one piece of contrasting material disposed over an outer surface of a proximal end of a lead. A piece of material can be contrasting when the piece of material is visually distinct, or conspicuous, from the portion of the lead on which the piece of contrasting material is disposed. In at least some embodiments, a piece of material disposed over an outer surface contrasts from the portion of the lead on which the material is disposed when the piece of material is a color that is different from the color of the portion of the lead on which the contrasting piece of material is disposed.

A piece of contrasting material can be formed in many different shapes, such as an oval, a circle, a rectangle, and the like. In a preferred embodiment, a piece of contrasting material is disposed over an outer surface of a proximal end of a lead and is formed as a contrasting band of material (“contrasting band”) that forms either a partial loop around at least a portion of a lateral circumference of a proximal end of a lead, or a complete loop around an entire lateral circumference of a proximal end of a lead.

The contrasting band can be formed in many different sizes. For example, the contrasting band may be formed in many different widths. The number of contrasting bands in a lead identifier may vary. For example, there can be one, two, three, four, five, six, or more contrasting bands in a lead identifier. As will be recognized, other numbers of contrasting bands may be used in a lead identifier. Each contrasting band can have the same or a different width when compared to other contrasting bands.

Contrasting bands can be formed from many different types of materials suitable for implantation that can be made visually distinct in appearance from a proximal end of a lead, including, plastic, metal, silicone, polyurethane, PEEK, epoxy, and the like or combinations thereof. Contrasting bands can be formed on an outer surface of a lead by any process, including heat shrinking colored tubing onto an outer surface of a lead, sliding colored tubing onto an outer surface of a lead, crimping or swaging contrasting bands onto a lead, or the like. Contrasting bands may be attached to an outer surface of a lead by an adhesive.

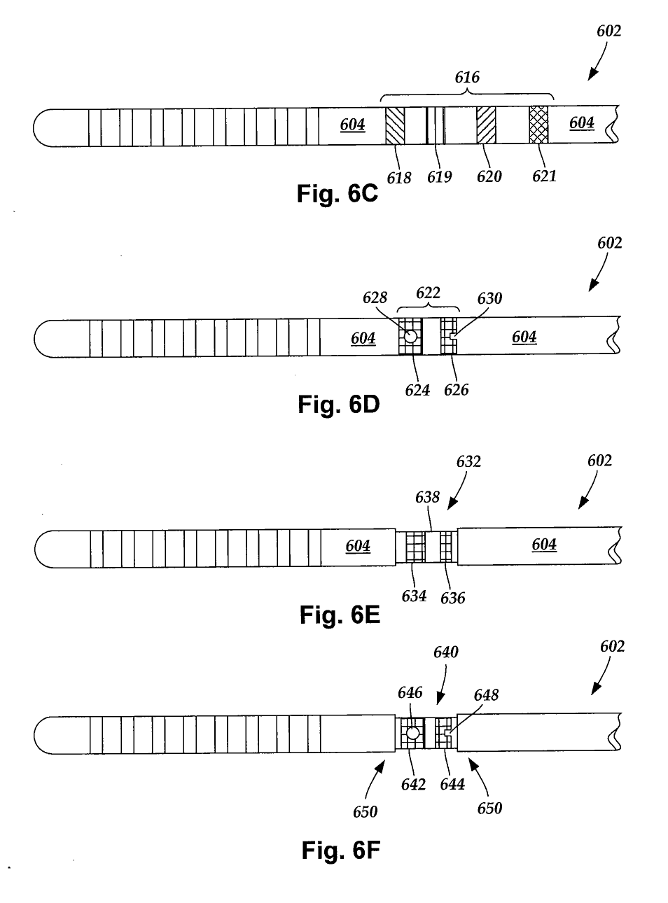

FIG. 6A is a schematic perspective view of one embodiment of a proximal end of the lead 602 with an outer surface 604. In FIG. 6A, a lead identifier 606 is shown as a contrasting band 608 wrapped over a portion of a lateral circumference of the outer surface 704 of the lead 702 without forming a complete loop. In at least some embodiments, the contrasting band 608 may be isodiametric, as shown in FIG. 6A. FIG. 6B is a schematic side view of one embodiment of a proximal end of a lead 602 with a lead identifier 610 that includes two contrasting bands 612 and 614 disposed over at least a portion of a lateral circumference of the proximal end of the lead 602. In FIG. 6C, the lead identifier 616 includes four contrasting bands 618-621 disposed on the outer surface 604 of the lead 602 that are each different in color and width from one another and are also each different in color from the outer surface 604 of the lead 602.

Additional markings can be used supplementally with one or more contrasting bands. Additional markings may include visible markings on, or in, the contrasting bands. For example, an additional marking may be one or more apertures, notches, grooves, protrusions, or the like formed in the band. FIG. 6D shows a schematic side view of one embodiment of a proximal end of a lead 602 that includes a lead identifier 622 with two contrasting bands 624 and 626 wrapped over at least a portion of a lateral circumference of the outer surface 604 of the lead 602. The two contrasting bands 624 and 626 each include a marking for further identification. The contrasting band 624 includes an aperture 628 and the contrasting band 626 includes a notch 630.

In alternate embodiments, lead identifiers that include one or more contrasting bands wrapped around at least a portion of an outer surface of a lead can be placed in an inset region in the outer surface of the lead. FIG. 6E is a schematic side view of one embodiment of a proximal end of a lead 602 that includes the outer surface 604. The lead 602 further includes a lead identifier 632 that includes two contrasting bands 634 and 636 wrapped over at least a portion of a lateral circumference of an inset region 638 in the outer surface 604 of the lead 602. FIG. 6F shows the lead 602 with a lead identifier 640 that includes two contrasting bands 642 and 644 with additional markings. The contrasting band 642 includes an aperture 646 and the contrasting band 644 includes a notch 648. In a preferred embodiment, contrasting bands placed within inset regions are isodiametric with adjacent portions 650 of the lead 602.

Various coding systems can be devised using the number of contrasting bands, the widths of the contrasting bands, the types of the materials used to form the contrasting bands, the colors of the contrasting bands, as well as any markings on the contrasting bands. The various coding systems can be used to provide information about the lead on which a lead identifier is disposed, such as the lead type, the number of proximal ends associated with the lead, which port a proximal end of a lead is to be inserted, the positioning of the distal end of the lead, or other information. For example, in one embodiment the number of contrasting bands corresponds to the number of proximal ends on a lead, the color of the contrasting bands corresponds to the type of lead, and the additional markings correspond to the specific ports of a connector into which each proximal end is to be inserted. In another example, one purple contrasting band may indicate that a proximal end of a lead is to be inserted into a first port on a control module and two purple contrasting bands may indicate that a proximal end of a lead is to be inserted into a second port of a control module. In yet another example, an odd number of green contrasting bands may indicate that a proximal end of a lead is to be inserted into a first port on a control module and an even number of green contrasting bands may indicate that a proximal end of a lead is to be inserted into a second port of a control module.

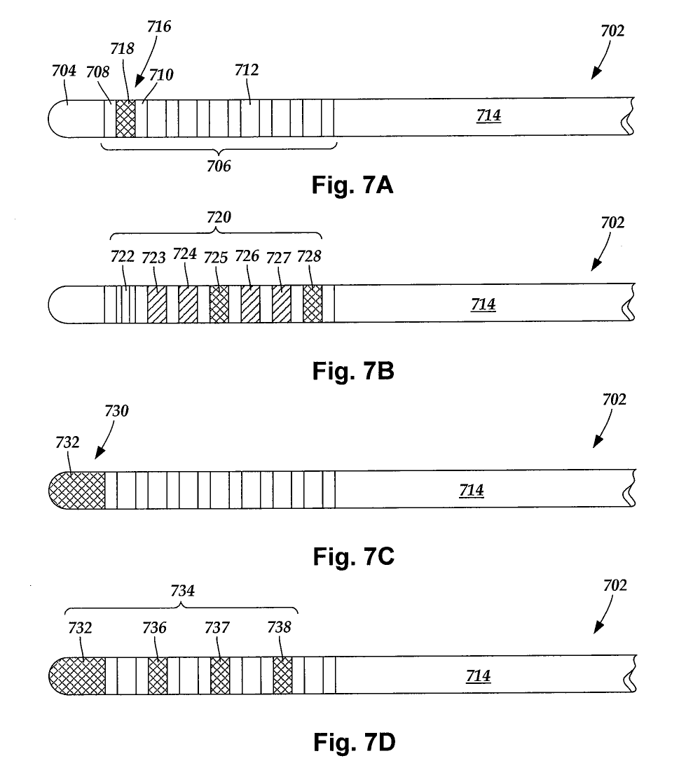

In at least some embodiments, a lead identifier includes one or more conspicuously-colored spacers disposed between adjacent terminals on a proximal end of a lead. In at least some embodiments, a spacer is conspicuously-colored when the spacer is a color that is different from the color of the remaining spacers, the proximal end of the lead, or the proximal tip. FIG. 7A is a schematic side view of one embodiment of a proximal end of a lead 702 with a proximal tip 704 and a plurality of terminals 706, such as adjacent terminals 708 and 710, separated from one another by spacers, such as spacer 712. The lead 702 further includes an outer lead covering 714. In at least some embodiments, the color of the lead body 714 is the same as the color of the proximal tip 704 and the spacers.

The lead 702 further includes a lead identifier 716 with a conspicuously-colored spacer 718 disposed between the two adjacent terminals 708 and 710. The color of the conspicuously-colored spacer 718 contrasts from the color of the lead body 714, the remaining spacers, and the proximal tip 704. Spacers can be formed from many different kinds of non-conductive, insulating materials suitable for implantation, including, silicone, polyurethane, PEEK, epoxy, and the like or combinations thereof. In at least some embodiments, the materials used to form a spacer are the same as the materials used to form the lead 702 or the proximal tip 704.

In at least some embodiments, a plurality of spacers are conspicuously-colored. FIG. 7B is a schematic side view of one embodiment of the proximal end of the lead 702 with a lead identifier 720 that includes seven conspicuously-colored spacers 722-728 each disposed between adjacent terminals. Each of the colors of the seven conspicuously-colored spacers 722-728 contrasts with the color of the proximal tip 704 and the lead body 714 of the lead 702. In alternate embodiments, each of the spacers 722-728 are the same contrasting color.

In at least some embodiments, different numbers, relative placement, or types of colors of spacers can be used in different arrangements to create various coding systems. The various coding systems can be used to provide information about the lead on which a lead identifier is disposed, such as the lead type, the number of proximal ends associated with the lead, which port a proximal end of a lead is to be inserted, the positioning of the distal end of the lead, or other information. For example, in one embodiment, a red spacer indicates that a proximal end of a lead corresponds with a first port of a control module. In another embodiment, two green spacers indicate that a proximal end of a lead corresponds with a second port of a control module. In yet another embodiment, a blue spacer indicates that a proximal end of a lead is the left lead body of a paddle body. In another embodiment, a white spacer indicates a right proximal end and a black spacer indicates a left proximal end of the lead. In another embodiment, one or more spacers with contrasting colors indicate a left proximal end and no spacers with contrasting colors indicates a right proximal end.

In at least some embodiments, a lead identifier includes a conspicuously-colored proximal tip on a proximal end of a lead. FIG. 7C is a schematic side view of one embodiment of the proximal end of the lead 702 with a lead identifier 730 that includes a conspicuously-colored proximal tip 732. In FIG. 7C, the color of the conspicuously-colored proximal tip 732 contrasts from the color of the lead body 714 of the lead 702 and any non-conspicuously-colored spacers. Proximal tips can be formed from many different kinds of non-conductive, insulating materials suitable for implantation, including, silicone, polyurethane, PEEK, epoxy, and the like or combinations thereof. In at least some embodiments, the materials used to form a proximal tip are the same as the materials used to form the lead 702 or the spacers.

In at least some embodiments, a lead identifier may include a conspicuously-colored proximal tip and one or more conspicuously-colored spacers. For example, FIG. 7D shows a schematic side view of one embodiment of the proximal end of the lead 702 with a lead identifier 734 that includes the conspicuously-colored proximal tip 732 and three conspicuously-colored spacers 736-738 each disposed between every other set of adjacent terminals. Many other combinations of conspicuously-colored spacers and the conspicuously-colored proximal tip 732 are possible. For example, in at least some embodiments a lead identifier of the proximal end of the lead 702 includes the conspicuously-colored proximal tip 732 and one conspicuously-colored spacer positioned between the two distal-most terminals. In FIG. 7D, the color of the conspicuously-colored proximal tip 732 contrasts from the color of the non-conspicuously-colored spacers and the lead body 714 of the lead 702. In some embodiments, the spacers 736-738 and the proximal tip 732 are the same contrasting color. In other embodiments, the spacers 736-738 are two or more different contrasting colors. In at least some embodiments, one or more conspicuously-colored spacers or the conspicuously-colored proximal tip is radiopaque.

In at least some embodiments, different numbers or types of colors of conspicuously-colored spacers and a conspicuously-colored proximal tip can be used in different arrangements to create various coding systems. For example, in one embodiment, a red proximal tip and a black spacer indicates that a proximal end of a lead corresponds with a first port of a control module. In another embodiment, two green spacers and a green proximal tip indicate that a proximal end of a lead corresponds with a second port of a control module. In yet another embodiment, a blue spacer and an orange proximal tip indicates that a proximal end of a lead is the left lead body of a paddle body.

In at least some embodiments, use of specific colors on spacers or a proximal tip (or pattern of spacers or number or arrangements of colors) corresponds to specific topographical information relating to the placement or orientation of a distal end of a lead. For example, in one embodiment, three brown spacers and a brown proximal tip may correspond to a proximal end of a lead with a distal end positioned in proximity to the lumbar vertebra of the patient's spine.

Sometimes connectors are formed out of translucent or transparent materials. Consequently, it may be difficult to insert a proximal end of a lead into an opening in a translucent or transparent connector during surgery. The embodiments shown in FIGS. 7A-7D additionally provide an advantage of facilitating insertion of a proximal end of a lead into a connector by providing a color on the proximal tip or one or more spacers between adjacent terminals that can be used to guide a proximal end of a lead into an opening of a connector or be visible inside the connector after insertion to provide a visual cue of when a proximal end of a lead is completely inserted into a connector.

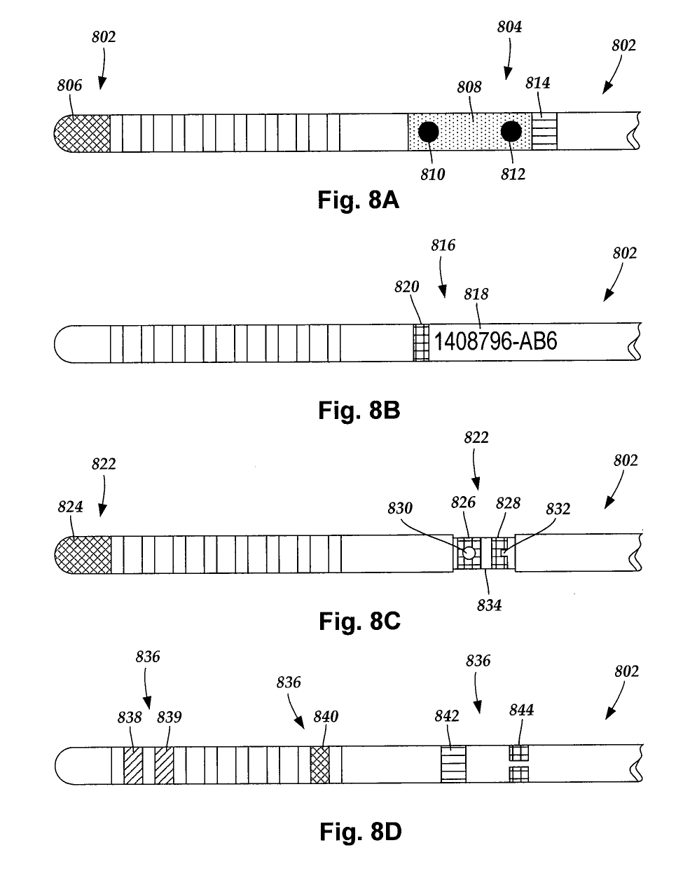

In at least some embodiments, a lead identifier includes various combinations of markable surface finishes, laser-ablated identification markings, contrasting bands wrapped around at least a portion of the proximal end of a lead, conspicuously-colored spacers, and a conspicuously-colored proximal tip. For example, FIG. 8A shows a schematic side view of one embodiment of a proximal end of a lead 802 with a lead identifier 804 that includes a conspicuously-colored proximal tip 806, a markable surface finish 808 marked with two dots 810 and 812, and a laser-ablated band 814. In another example, FIG. 8B shows a schematic side view of one embodiment of the proximal end of the lead 802 with a lead identifier 816 that includes a laser-ablated identification code 818 and a contrasting band 820 wrapped around at least a portion of the lead 802. In yet another example, FIG. 8C shows a schematic side view of one embodiment of the proximal end of the lead 802 with a lead identifier 822 that includes a conspicuously-colored proximal tip 824 and two contrasting bands 826 and 828 with additional markings 830 and 832, respectively, wrapped over at least a portion of the circumference of an inset region 834 disposed in the lead 802. In another example, FIG. 8D shows a schematic side view of one embodiment of the proximal end of the lead 802 with a lead identifier 836 that includes three conspicuously-colored spacers 838-840, a laser-ablated band 842, and contrasting band 844 wrapped around a portion of the circumference of the proximal end of the lead 802.

In at least some embodiments, one or more coding systems may be created using various combinations of markable surface finishes, laser-ablated identification markings, contrasting bands wrapped around at least a portion of the proximal end of a lead, conspicuously-colored spacers, and conspicuously-colored proximal tips. Various combinations and arrangements can be used to provide various types of information about the lead on which the lead identifier is disposed, such as the type of lead, the number of proximal ends associated with the lead, the number of leads implanted in a patient, the location of the distal end of the lead on which the lead identifier is disposed, or any special instructions related to a specific lead.

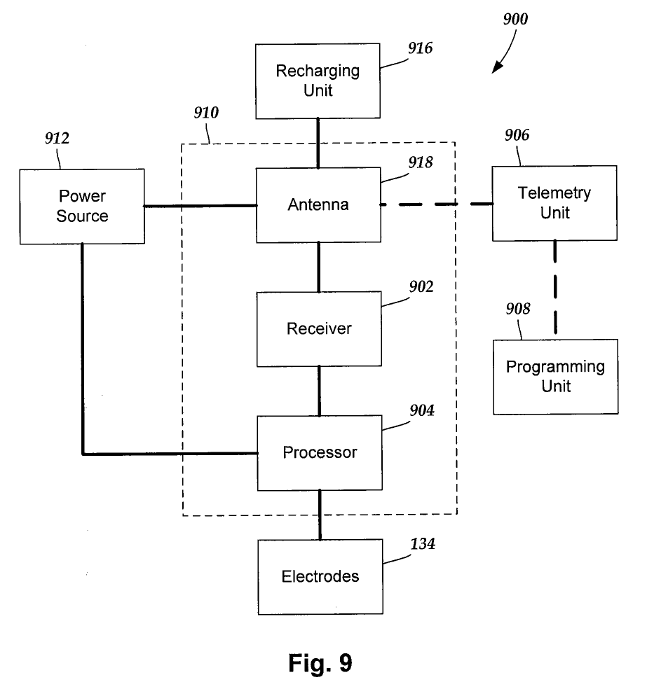

FIG. 9 is a schematic overview of one embodiment of components of an electrical stimulation system 900 including an electronic subassembly 910 disposed within a control module. It will be understood that the electrical stimulation system can include more, fewer, or different components and can have a variety of different configurations including those configurations disclosed in the stimulator references cited herein.

Some of the components (for example, power source 912, antenna 918, receiver 902, and processor 904) of the electrical stimulation system can be positioned on one or more circuit boards or similar carriers within a sealed housing of an implantable pulse generator, if desired. Any power source 912 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Patent Application Publication No. 2004/0059392, incorporated herein by reference.

As another alternative, power can be supplied by an external power source through inductive coupling via the optional antenna 918 or a secondary antenna. The external power source can be in a device that is mounted on the skin of the user or in a unit that is provided near the user on a permanent or periodic basis.

If the power source 912 is a rechargeable battery, the battery may be recharged using the optional antenna 918, if desired. Power can be provided to the battery for recharging by inductively coupling the battery through the antenna to a recharging unit 916 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 904 is generally included to control the timing and electrical characteristics of the electrical stimulation system. For example, the processor 904 can, if desired, control one or more of the timing, frequency, strength, duration, and waveform of the pulses. In addition, the processor 904 can select which electrodes can be used to provide stimulation, if desired. In some embodiments, the processor 904 may select which electrode(s) are cathodes and which electrode(s) are anodes. In some embodiments, the processor 904 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 908 that, for example, allows modification of pulse characteristics. In the illustrated embodiment, the processor 904 is coupled to a receiver 902 which, in turn, is coupled to the optional antenna 918. This allows the processor 904 to receive instructions from an external source to, for example, direct the pulse characteristics and the selection of electrodes, if desired.

In one embodiment, the antenna 918 is capable of receiving signals (e.g., RF signals) from an external telemetry unit 906 which is programmed by a programming unit 908. The programming unit 908 can be external to, or part of, the telemetry unit 906. The telemetry unit 906 can be a device that is worn on the skin of the user or can be carried by the user and can have a form similar to a pager, cellular phone, or remote control, if desired. As another alternative, the telemetry unit 906 may not be worn or carried by the user but may only be available at a home station or at a clinician's office. The programming unit 908 can be any unit that can provide information to the telemetry unit 906 for transmission to the electrical stimulation system 900. The programming unit 908 can be part of the telemetry unit 906 or can provide signals or information to the telemetry unit 906 via a wireless or wired connection. One example of a suitable programming unit is a computer operated by the user or clinician to send signals to the telemetry unit 906.

The signals sent to the processor 904 via the antenna 918 and receiver 902 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 900 to cease operation, to start operation, to start charging the battery, or to stop charging the battery. In other embodiments, the stimulation system does not include an antenna 918 or receiver 902 and the processor 904 operates as programmed.

Optionally, the electrical stimulation system 900 may include a transmitter (not shown) coupled to the processor 904 and the antenna 918 for transmitting signals back to the telemetry unit 906 or another unit capable of receiving the signals. For example, the electrical stimulation system 900 may transmit signals indicating whether the electrical stimulation system 900 is operating properly or not or indicating when the battery needs to be charged or the level of charge remaining in the battery. The processor 904 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A lead comprising:
a lead body with a distal end and at least one proximal end;
a plurality of electrodes disposed on the distal end of the lead body;
a plurality of terminals disposed on the at least one proximal end of the lead body;
a plurality of conductor wires extending along the lead body to couple the electrodes electrically to the terminals; and
at least one lead identifier disposed on the lead body, the lead identifier configured and arranged to visually identify the end of the lead body on which the at least one lead identifier is disposed, the at least one lead identifier comprising at least one of
a markable-surface-finish region suitable for marking with a pen,
at least one laser-ablated identification marking,
at least one contrasting band of material formed of a conspicuous color,
at least one conspicuously-colored spacer disposed between two adjacent terminals of at least one of the at least one proximal end of the lead body, or
a conspicuously-colored proximal tip disposed on at least one of the at least one proximal end of the lead body.

2. The lead of claim 1, wherein the at least one contrasting band of material is wrapped around at least a portion of a lateral circumference of the at least one proximal end of the lead.

3. The lead of claim 1, wherein the at least one contrasting band of material includes at least one additional marking.

4. The lead of claim 3, wherein the at least one additional marking is at least one of a notch, an aperture, a groove, or a protrusion.

5. The lead of claim 1, wherein the at least one contrasting band of material is inset in a recess disposed in the outer surface of at least one of the at least one proximal end of the lead.

6. The lead of claim 1, wherein the contrasting band of material comprises heat shrink tubing.

7. The lead of claim 1, wherein the contrasting band of material comprises at least one piece of metal.

8. The lead of claim 7, wherein the at least one piece of metal is swaged.

9. The lead of claim 1, wherein the at least one laser-ablated identification marking is at least one of an identification code or a band.

10. The lead of claim 1, wherein the lead identifier is further configured and arranged to identify the location of the distal end of the lead when the distal end of the lead is implanted in a patient.

11. The lead of claim 1, wherein the lead identifier is radiopaque.

12. The lead of claim 1, wherein the markable-surface-finish region is formed by laser ablating a portion of the at least one proximal end of the lead.

13. An electrical stimulating system comprising:
a first lead comprising
a lead body with a distal end and at least one proximal end;
a plurality of electrodes disposed on the distal end of the lead body;
a plurality of terminals disposed on the at least one proximal end of the lead body;
a plurality of conductor wires extending along the lead body to couple the electrodes electrically to the terminals; and
at least one lead identifier disposed on the lead body, the lead identifier configured and arranged to visually identify the end of the lead body on which the at least one lead identifier is disposed, the at least one lead identifier comprising at least one of
a markable-surface-finish region suitable for marking with a pen,
at least one laser-ablated identification marking,
at least one contrasting band of material formed of a conspicuous color,
at least one conspicuously-colored spacer disposed between two adjacent terminals of at least one of the at least one proximal end of the lead body, or
a conspicuously-colored proximal tip disposed on at least one of the at least one proximal end of the lead body;
a control module configured and arranged to electrically couple to the first lead, the control module comprising
a housing,
an electronic subassembly disposed in the housing; and
a connector for receiving the first lead, the connector comprising
a connector housing defining at least one opening for receiving the at least one proximal end of the first lead, and
a plurality of connector contacts disposed in the connector housing, the connector contacts configured and arranged to couple to at least one terminal on the at least one proximal end of the first lead.

14. The electrical stimulating system of claim 13, further comprising a second lead comprising
a lead body with a distal end and at least one proximal end;
a plurality of electrodes disposed on the distal end of the lead body;
a plurality of terminals disposed on the at least one proximal end of the lead body;
a plurality of conductor wires extending along the lead body to couple the electrodes electrically to the terminals; and
at least one lead identifier disposed on the lead body, the lead identifier configured and arranged to visually identify the end of the lead body on which the at least one lead identifier is disposed, the at least one lead identifier comprising at least one of
a markable-surface-finish region suitable for marking with a pen,
at least one laser-ablated identification marking,
at least one contrasting band of material formed of a conspicuous color,
at least one conspicuously-colored spacer disposed between two adjacent terminals of at least one of the at least one proximal end of the lead body, or
a conspicuously-colored proximal tip disposed on at least one of the at least one proximal end of the lead body,
wherein the control module is configured and arranged to electrically couple to the second lead and the at least one lead identifier of the second lead is different from the at least one lead identifier of the first lead.

15. The electrical stimulating system of claim 13, further including a lead extension having a proximal end and a distal end, the connector disposed on the distal end of the lead extension.

16. The electrical stimulating system of claim 15, wherein the proximal end of the lead extension is configured and arranged for insertion into another connector.

17. The electrical stimulating system of claim 13, wherein the connector is disposed on a control module.

18. The electrical stimulating system of claim 13, wherein the connector is disposed on at least one of an operating room cable or an adaptor configured and arranged for electrical coupling to an electronic device.

19. A method for stimulating patient tissue, the method comprising:
implanting at least one lead into a patient, the at least one lead comprising a plurality of electrodes disposed on a distal end of the at least one lead and electrically coupled to a plurality of terminals disposed on each of at least one proximal ends of each of the at least one lead, the lead comprising at least one lead identifier for individually identifying each of the at least one proximal ends of the at least one lead, the at least one lead identifier comprising at least one of
a markable-surface-finish region suitable for marking with a pen,
at least one laser-ablated identification marking,
at least one contrasting band of material formed of a color that is different from the first color,
at least one conspicuously-colored spacer disposed between two adjacent terminals of at least one of the at least one proximal end of the lead body, the at least one conspicuously-colored spacer formed of a color that is different from the first color, or
a conspicuously-colored proximal tip disposed on at least one of the at least one proximal end of the lead body, the at least one conspicuously-colored proximal tip formed of a color that is different from the first color;
disposing each of the at least one proximal ends of the at least one lead into at least one connector, each of the at least one connector defining at least one port for receiving the at least one proximal ends of the at least one lead, each of the at least one port comprising a plurality of connective contacts that electrically couple to at least one of the plurality of terminals disposed on each of at least one proximal ends of each of the at least one lead, each of the at least one the connectors electrically coupled to at least one control module; and
providing electrical signals from at least one of the control modules to electrically stimulate patient tissue using at least one of the electrodes on at least one of the at least one lead.

20. The method of claim 19, wherein implanting at least one lead into a patient comprises implanting a plurality of the leads into the patient, wherein the at least one lead identifier of each of the leads is different.
